(19) United States
(12) Patent Application Publication
(10) Pub. No.: US 2008/0063605 A1
(43) Pub. Date: Mar. 13, 2008

(54) Method of Enhancing Visualization of Atherosclerotic Plaque

(76) Inventors: Wai-Fung Cheong, Los Altos, CA (US); Hugo Madden, Palo Alto, CA (US); Greg W. Hemmi, Sunnyvale, CA (US)

Correspondence Address: WILSON SONSINI GOODRICH & ROSATI, 650 PAGE MILL ROAD, PALO ALTO, CA, 94304-1050, US

(21) Appl. No.: 11/579,759

(22) PCT Filed: May 19, 2005
(86) PCT No.: PCT/US05/17812

§ 371 Date: Aug. 24, 2007

Related U.S. Application Data
(60) Provisional application No. 60/573,397, filed on May 20, 2004.

(51) Int. Cl. A61B 5/055

(52) U.S. Cl. 424/9.363

(57) ABSTRACT

The invention relates to a method and a corresponding device for multipurposely supporting a randomly shaped and/or possibly complex with respect to a rigid base (11) part (1) by fixing a working reference, wherein the inventive method consist in interposing at least one deformable constant-volume chamber (3) which is sealed and filled with incompressible particles (4) between a maintainable part and the rigid base (11) and in lowing pressure inside the deformable constant-volume chamber (3) by means of a vacuum source (5) connectable thereto, thereby enabling the particles (4) to be amalgamated into a solid block which thrusts at least partly on the rigid base (11) and maintains the part (1) by fitting the shape thereof.

### CLAIM OF PRIORITY

This Application claims priority from U.S. Provisional Patent Application No. 60/573,397, the contents of which are incorporated herein in their entirety.

### FIELD OF INVENTION

The present invention relates to a method of enhancing visualization of atherosclerotic plaque by using a compound of Formula I.

### BACKGROUND OF THE INVENTION

Atherosclerosis is now established as a chronic inflammatory disease wherein certain lesions (example vulnerable plaque, VP) at highest risk for initiating lethal acute heart attacks are unstable, diffuse, and characterized by a lipid-rich core of inflammatory cells beneath a thin fibrous cap. Poor hemodynamics and actions by enzymes, e.g., matrix metallo-proteinases, gradually degrade cap stability, making it vulnerable to rupture. When the rupture happens, highly inflammatory material spills from the core into the vessel lumen to form blood clots, occlude flow, and induce an infarct. Prior to plaque rupture, patients with such vulnerable lesions may be asymptomatic. Angiography fails to detect them because the characteristic narrowing associated with stable lesions is often not seen in angiograms of vulnerable plaques.

Modalities to image or detect vulnerable plaque (VP) must therefore provide both anatomic and molecular (i.e., functional) information about the lesions and vessel wall. Techniques, both noninvasive and interventional, are being researched and developed. Among these are multislice CT scanning, magnetic resonance imaging (MRI), intravascular ultrasound (radiofrequency IVUS, elastography), optical methods (Optical Coherence Tomography, NIR spectroscopy, Mid-Infrared imaging), and thermography. Some of these techniques need to be enhanced with intravenous administration of exogenous compounds that target specific components of VP (e.g., lipids, foam cells or macrophages) and are themselves detectable because of specific physical properties (e.g., paramagnetism, acoustic impedances, index of refraction). There is thus a continuing need for methods and compounds that can be used to visualize arterial plaque.

### SUMMARY OF THE INVENTION

The present invention provides a method for enhancing visualization of plaque, said method comprising:

(a) administering to a host a compound of Formula I: 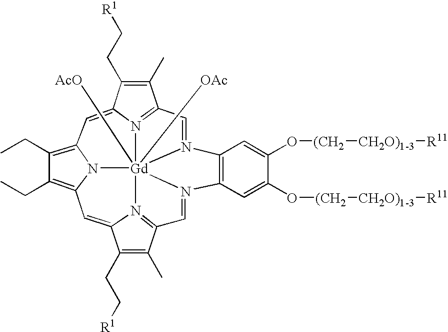
wherein:
R¹ independently at each occurrence represents —CH₂OH, —CH₂O(C═O)CH₃, or —C(═O)OCH₃; and
R¹¹ independently at each occurrence represents H or —CH₃; and

(b) imaging said host using magnetic resonance imaging (MRI).

### DETAILED DESCRIPTION

The present invention provides a method for enhancing visualization of atherosclerotic plaque, said method essentially comprising:

(a) administering to a host a compound of Formula I: 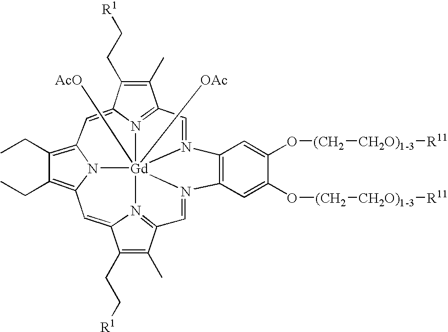
wherein:
R¹ independently at each occurrence represents —CH₂OH, —CH₂O(C═O)CH₃, or —C(═O)OCH₃; and
R¹¹ independently at each occurrence represents H or —CH₃; and

(b) imaging said host using magnetic resonance imaging (MRI).

A preferred embodiment of the present invention provides a method wherein the atherosclerotic plaque being imaged is arterial atherosclerotic plaque. Another preferred embodiment provides a method wherein the compound of Formula I is

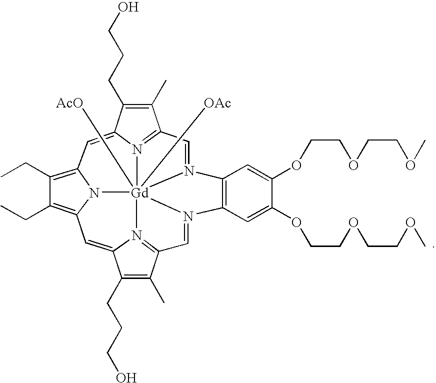

Another preferred embodiment provides a method wherein the compound of Formula I is:

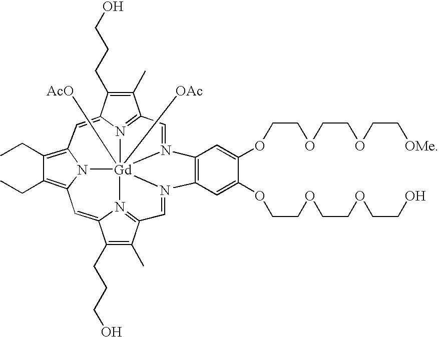

Yet another preferred embodiment is a method wherein the compound of Formula I is:

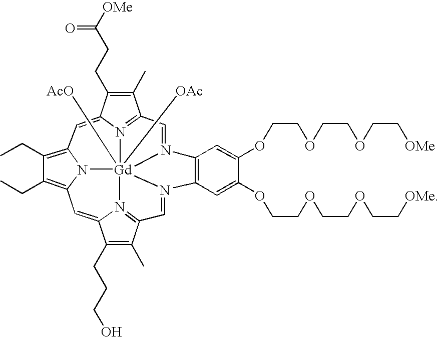

Provided in yet another preferred embodiment is a method wherein the compound of Formula I is:

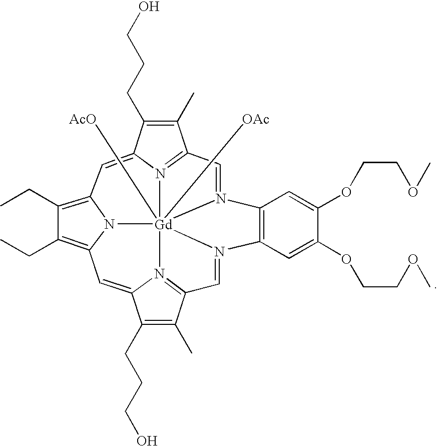

A further preferred embodiment of the present invention provides a method wherein the compound of Formula I is:

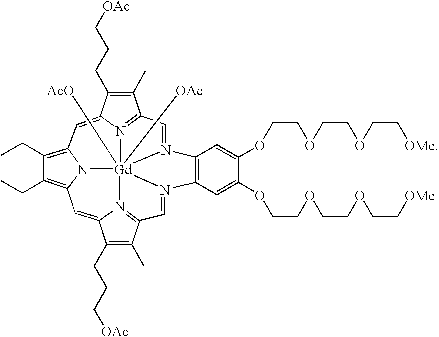

### FIGURES

FIG. 1: This figure shows MRI images obtained using compound in Ex. 2 on a Watanabe Hereditable Hyperlipidemic (WHHL) rabbit model of atherosclerosis, highlighting the plaque area. In particular the lipid core within the plaque area is highlighted.

FIG. 2: This figure shows confocal images of endothelial cells exposed to 50 uM of the compound of Formula I (left), unexposed cells (middle), and smooth muscle cell exposed to 100 uM of the compound of Formula I (right). Pseudo-red color indicates fluorescence

FIG. 3: This figure shows T1-weighted images of an atheroma acquired at the same plane at different times post-administration of the compound in Ex. 2. SNR and CNR curves are included. Enhancement of plaque features, e.g., protrusion into the lumen and the cap overlying a lipid core (see arrow), which were not visible at baseline, became visible over time.

### EXPERIMENTAL

Compounds of Formula I can be synthesized by procedures known to one skilled in the art. One such procedure is as outlined in U.S. Pat. No. 5,994,353. The following examples were prepared using the above procedure:

### EXAMPLE 1

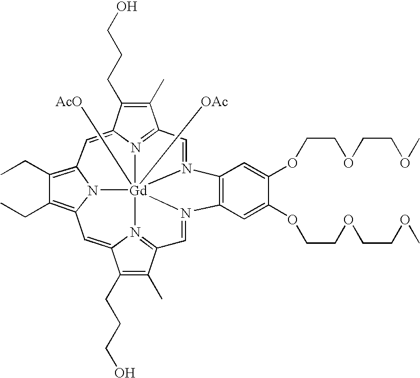

### EXAMPLE 2

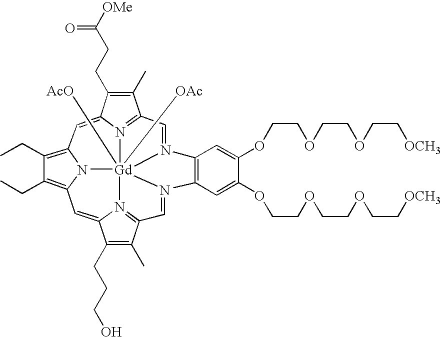

### EXAMPLE 3

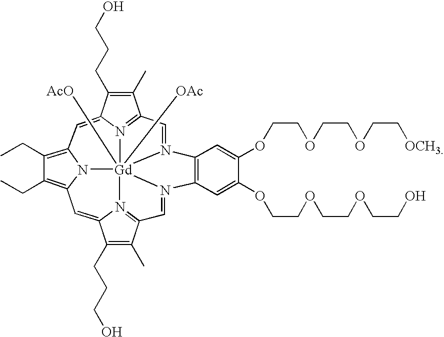

### EXAMPLE 4

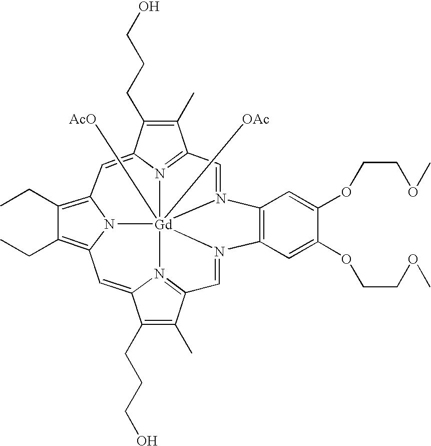

### EXAMPLE 5

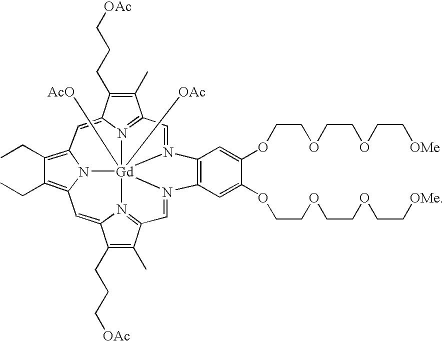 Compound Administration

Watanabe Hereditable Hyperlipidemic (WHHL) rabbits received focal injury in their sub-renal abdominal aorta, and were kept on a high-cholesterol diet for at least 6-8 weeks before imaging studies began.

The Formula I compounds (Examples 1, 2, 3 and 4) were formulated in 5% mannitol at concentrations of 2 mg/mL and administered intravenously at a dose of 10 mg/kg. At any one time, only one compound was injected into a rabbit. Some rabbits were occasionally re-scanned by injecting a different Formula I compound with at least a week elapsing between scans. Scanning was done on a 1.5 Tesla MRI System (Philips Medical) with two 10 cm phase array surface coils. Multiple sub-renal ECG-gated fat-saturation aortic 3D-black-blood Fast-Spin-Echo vessel wall images (TR=3 RR, TE=10.5 ms, TI pre/post=400/280 ms, FOV=76 mm, in-plane resolution=250 μm) were acquired pre- and post-administration of a compound of Formula I (10 mg/kg i.v. per animal) and every 10 minutes over 120 minutes using 2 mm slice thickness. Signal-to-noise ratio (SNR) and Contrast-to noise ratio (CNR) were characterized over time using a semi-automated analysis algorithm to examine the effects of aortic wall pharmacokinetics/dynamics of the compounds on the images acquired.

Once the compound of Formula I had been taken up by plaques, it altered the proton relaxivity of water associated with or in close proximity to the gadolinium metal in the complex; and its presence within the cells enhanced detectability of the plaque under MRI.

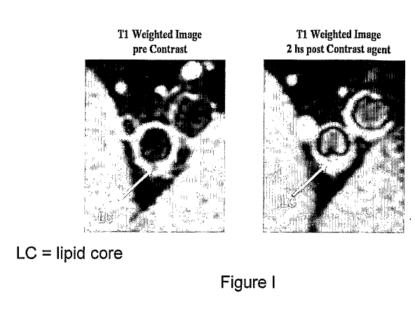

Formula I compounds facilitate preferential imaging of plaque, such as vulnerable plaque, as shown in FIG. 1 and Table 1

TABLE 1
Percent increases from baseline to 60 min post-administration
of Formula I compound in T1-weighted signal-to-noise (T1-SNR)
and contrast-to-noise (T1-CNR) for three Gd-Tex complexes.

| Gd-Tex | | |
|---|---|---|
| complex | T1-SNR | T1-CNR |
| Ex. 2 | 75% | 43% |
| Ex. 3 | 20% | Unchanged ⁽*⁾ |
| Ex. 4 | 8% | 6% |

⁽*⁾ The T1-CNR for Ex. 3 had its peak contrast at 15-30 min (˜50% improvement).

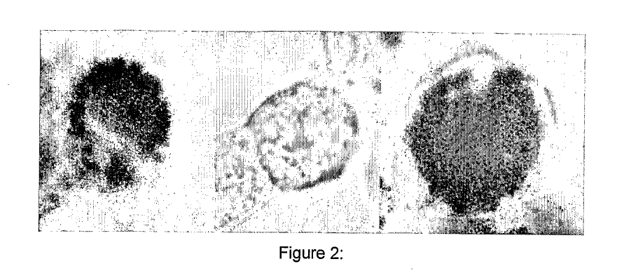

Compounds of Formula I localize in intracellular spaces as shown in FIG. 2. The uptake of Formula I compounds by cells is gradual and over a period of time. Once inside a cell, compounds of Formula I seem to have a prolonged residency within the cell thereby providing an opportunity to image the cell over a prolonged period of time and at different time intervals.

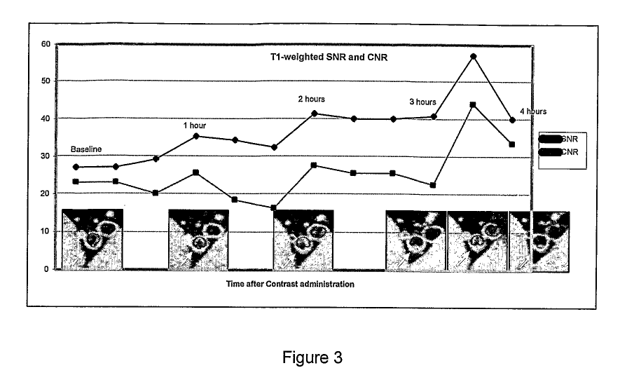

The slower pharmacokinetics and higher cellular selectivity of compounds of Formula I make sequential magnetic resonance (MR) imaging of the target tissue (plaque) possible. MR images are collected as Formula I compounds are taken up and cleared from the target tissue, thereby providing a composite molecular picture of the tissue or lesion. The pharmacokinetics and target tissue selectivity is affected by the nature of the Formula I compound, its formulation, and the imaging sequence used (e.g., T1-weighted, T2-weighted, Proton Density Weighted, FSE, TR, TE). Thus, for example, lipids appear hyperintense under T1W protocol but hypointense under T2W protocol. FIG. 3 provides one such dynamic approach—the same lesion looks different at different times because of the drug pharmacokinetics.

Abbreviations

OAc: —O—C(═O)—CH₃

1. A method of enhancing visualization of plaque, said method essentially comprising:
(a) administering to a host a compound of Formula I: 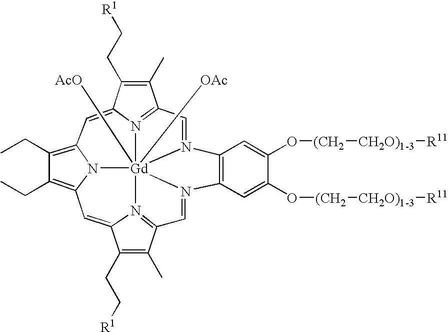
wherein:
R¹ independently at each occurrence represents —CH₂OH, —CH₂O(C═O)CH₃, or
—C(═O)OCH₃; and
R¹¹ independently at each occurrence represents H or —CH₃; and
(b) imaging said host using magnetic resonance imaging (MRI).

2. A method of claim 1 wherein, the plaque being imaged is arterial atherosclerotic plaque.

3. A method of claim 2 wherein the compound of Formula I is

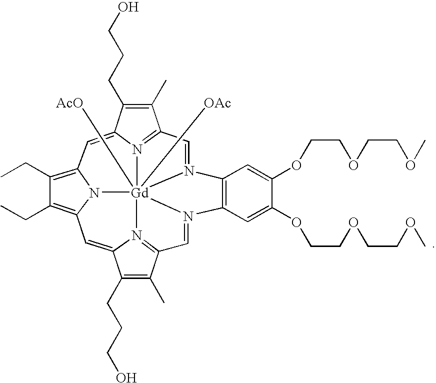

4. A method of claim 2 wherein the compound of Formula I is:

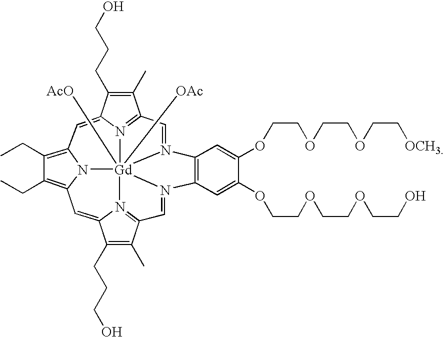

5. A method of claim 2 wherein the compound of Formula I is:

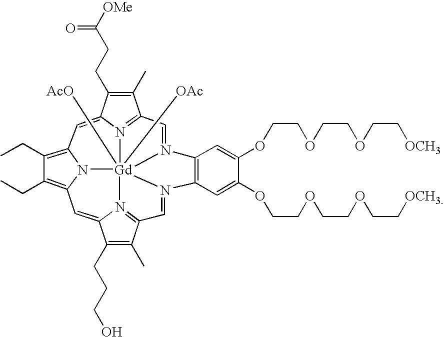

6. A method of claim 2 wherein the compound of Formula I is:

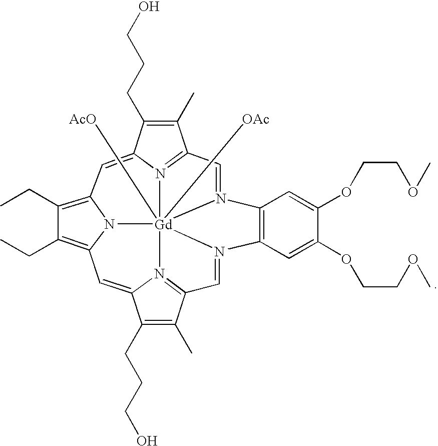

7. A method of claim 2 wherein the compound of Formula I is

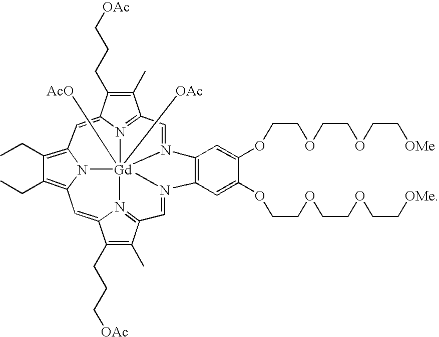

8. A method of enhancing visualization of plaque, said method essentially comprising:
(a) administering to a host a compound of Formula I: 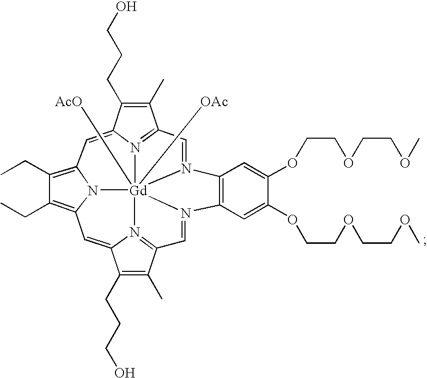
and
(b) imaging said host using magnetic resonance imaging (MRI).

9. A method of enhancing visualization of plaque, said method essentially comprising:
(a) administering to a host a compound of Formula I: 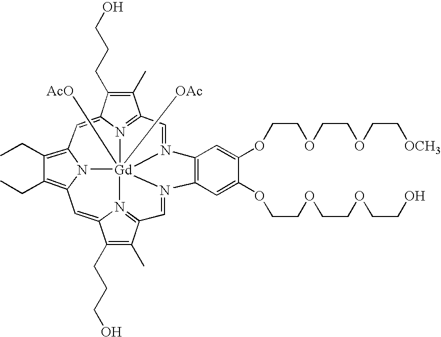
and
(b) imaging said host using magnetic resonance imaging (MRI).

10. A method of enhancing visualization of plaque, said method essentially comprising:
(a) administering to a host a compound of Formula I: 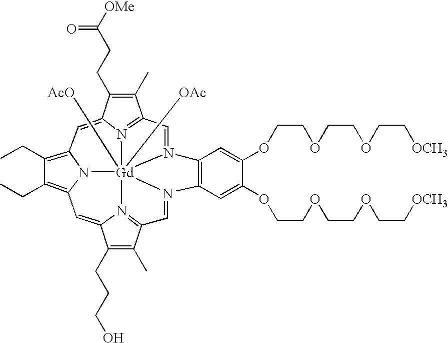
and
(b) imaging said host using magnetic resonance imaging (MRI).

11. A method of enhancing visualization of plaque, said method essentially comprising:
(a) administering to a host a compound of Formula I: 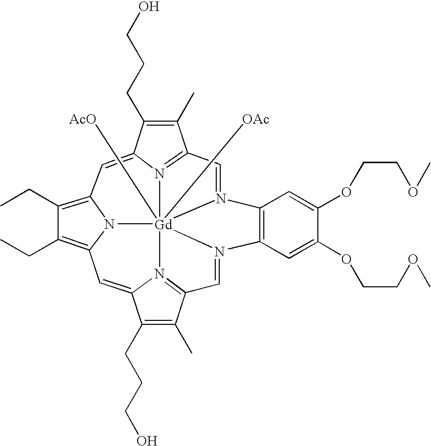
and
(b) imaging said host using magnetic resonance imaging (MRI).

12. A method of enhancing visualization of plaque, said method essentially comprising:
(a) administering to a host a compound of Formula I: 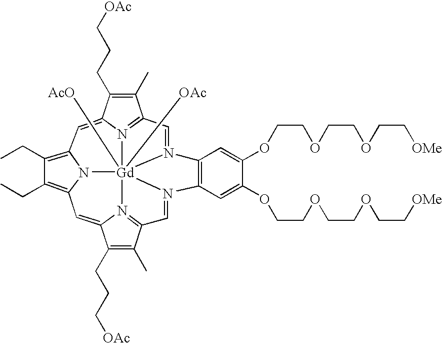
and
(b) imaging said host using magnetic resonance imaging (MRI).
